(19) United States
(12) Patent Application Publication
(10) Pub. No.: US 2014/0288409 A1
(43) Pub. Date: Sep. 25, 2014

(54) MAGNETIC RESONANCE DEVICE FOR SCANNING AN EXTREMITY OF A PATIENT

(71) Applicant: SIEMENS AKTIENGESELLSCHAFT, Munchen (DE)

(72) Inventors: Patrick GROSS, Ismaning (DE); Sebastian SCHMIDT, Weisendorf (DE)

(73) Assignee: SIEMENS AKTIENGESELLSCHAFT, Munchen (DE)

(21) Appl. No.: 14/220,333

(22) Filed: Mar. 20, 2014

(30) Foreign Application Priority Data
Mar. 25, 2013 (DE) 102013205213.8

(51) Int. Cl. A61B 5/055

(52) U.S. Cl. CPC A61B 5/0555
USPC 600/415

(57) ABSTRACT

A magnetic resonance device for scanning an extremity of a patient, in particular a leg, comprising a housing and an extremity radiography unit containing an imaging region, into which the extremity can be introduced when patients situated outside the extremity radiography unit, in which case the extremity radiography unit is arranged along a horizontal transverse axis perpendicular to its longitudinal axis toward one side of the housing, in which case a magnet unit forming the extremity radiography unit is configured to be capable of pivoting around a horizontal pivot axis perpendicular to the longitudinal axis by means of a pivot mechanism at a certain angular interval located about a horizontal situation of the longitudinal axis is provided.

### CROSS-REFERENCE TO RELATED APPLICATIONS

This application claims priority to DE Application No. 102013205213.8, having a filing date of Mar. 25, 2013, the entire contents of which are hereby incorporated by reference.

### FIELD OF TECHNOLOGY

The following relates to a magnetic resonance device for scanning an extremity of a patient, in particular a leg, comprising a housing and an extremity radiography unit containing the imaging region, into which the extremity can be introduced in the case of patients situated outside the extremity radiography unit, in which case the extremity radiography unit is arranged along a horizontal transverse axis perpendicular to its longitudinal axis toward one side of the housing.

### BACKGROUND

Magnetic resonance devices and their functionality are already widely known in the prior art. Magnetic resonance devices are frequently intended for an entire person, which means that they have a large patient receiving cavity formed by a main magnet unit. Such whole-body magnetic resonance devices require a large amount of space, which must be shielded, and tend to be expensive to purchase. It has therefore been proposed to create smaller magnetic resonance devices for special fields of application which can be produced in a smaller physical size and having lesser requirements in terms of shielding cabins and the like.

An example of such types of dedicated magnetic resonance devices are so-called extremity scanners, in other words magnetic resonance devices for scanning an extremity of a patient. These are frequently employed in order to produce magnetic resonance images in the region of a leg of a patient, for example, magnetic resonance images of the knee of a patient. In such types of magnetic resonance devices for scanning an extremity of a patient only the target anatomy, whether it be an arm or a leg, needs to be arranged within the extremity radiography unit. For this it is known to integrate the magnet unit and consequently also the extremity radiography unit into the overall housing, in which case the extremity radiography unit is not arranged centrally in the overall housing containing, for example, electronics components and the like but is arranged mostly toward one side. On the other hand, it is known to position the extremity radiography unit angled downward from the horizontal in its longitudinal direction, thereby enabling a comfortable patient position, for example, in the situation when the patient sits on a dedicated chair in front of the magnetic resonance device.

This embodiment has several disadvantages. On the one hand, a scan of the left leg can be configured very conveniently for example in the situation when the extremity radiography unit is first arranged on the right-hand side of the housing because the right leg can be placed beside the housing. However, if the right leg, for example, the right knee, is to be examined the examination is rendered considerably more difficult because the patient must place his left leg around the housing, which extends further in this direction, and must consequently splay his leg wide. It should be noted in this situation that the tilted embodiment of the extremity radiography unit makes it impossible to insert the leg into the extremity radiography unit from the other side of the magnetic resonance device since it would then lie angled upward, which is very uncomfortable. An examination from the rear side of the magnetic resonance device also conflicts with the fact that the space available in hospitals and medical practices using such a type of extremity scanner is for the most part reduced, in which case it should additionally be noted that one advantage of the magnetic resonance devices for scanning extremities is precisely the fact that it has a small space requirement. Said advantage would be negated if it were necessary to make access possible from the front and the rear. Under these circumstances, it is in some cases even totally impossible to carry out certain examinations for patients having restricted mobility.

A known magnetic resonance device of the type described is obtainable for example under the product name “GE Optima MR430S” from the General Electric Company, GE Healthcare Division. This is available for strong fields, specifically for example 1.5 Tesla.

### SUMMARY

An aspect relates to an extremity magnetic resonance device such that user convenience and the fields of application are enhanced.

In the case of a magnetic resonance device of the type mentioned in the introduction, provision is made for a magnet unit forming the extremity radiography unit to be designed to be capable of pivoting around a horizontal pivot axis perpendicular to the longitudinal axis by means of a pivot mechanism at a certain angular interval located about a horizontal situation of the longitudinal axis.

It is consequently proposed to no longer integrate the magnet unit completely into the housing but to mount same capable of pivoting in said housing in order to thus be able to assign different pivot positions to different task requirements during the examination. The magnet unit is therefore, implemented by means of the pivot mechanism, mounted so as to be capable of pivoting in the housing, namely as has been described still also toward one side of the housing which in fact exhibits a certain minimal width. If the magnet unit when viewed for example from a front side is arranged more to the right, in the situation when the magnet unit with the extremity radiography unit is tilted slightly backward this enables a left leg of a patient to be scanned extremely comfortably, in which case the possible field of application as has already been described in the prior art is given in said configuration. As a result of the fact that the magnet unit and thus the extremity radiography unit can however be tilted, the additional possibility now presents itself for example of tilting the magnet unit forward and for example introducing a right leg into the extremity radiography unit from the rear, which means that the left leg can then be arranged comfortably beside the housing which is not very wide there. In particular, provision can be made that the outer edge of the magnet unit toward one side of the housing also forms an outer edge of the magnetic resonance device overall, and therefore no further housing components whatsoever are present there beside the magnet unit.

It has been recognized that at least in the case of smaller magnetic resonance devices intended for extremities the magnet unit can be tilted or pivoted in a manner which is really simple to implement because the magnet unit, comprising the in particular superconducting main magnet, a gradient coil arrangement and a high-frequency coil arrangement, has a low weight. This provides the opportunity to carry out a comfortable and flexible magnetic resonance examination of extremities. The magnetic resonance device can be used from both sides.

It is particularly preferred in at least one situation if the main field strength of the magnetic resonance device is ≧1 Tesla and/or an actively screened, a superconducting main magnet is used. High-quality magnetic resonance images can then be produced.

For example, provision can be made for the angular interval to be 20°. Tilting from the horizontal by up to 10° in each case can then take place. Tilting from the horizontal by 10° enables the extremity, in particular the leg, to be supported particularly comfortably, in which case tilting by a small angular interval such as 20° can be easily implemented.

The pivot mechanism can comprise a guiding assembly having a plurality of engagement points for different pivot positions. The magnet unit can therefore be guided inside the housing, in which case a plurality of engagement points for predetermined, expedient pivot positions is present. For example, in the stated example of an angular interval of 20° provision can be made to provide engagement points at −10°, 0° and 10°. Generally speaking it is consequently expedient if engagement points are present for a central position in which the longitudinal axis lies in the horizontal plane and for at least two inclined positions lying in particular at the edge of the angular interval.

It is expedient in this context if connection facilities for signal lines and power supply lines to the magnet unit are arranged at the engagement points, comprising, in particular, at least one sliding contact and/or at least one plug-in location forming part of an engagement assembly defining the engagement point and/or at least one wireless connection facility. Wireless connection facilities are suitable in particular in the situation when signals having a rather low energy level are to be transmitted. For power supply lines, an implementation using a sliding contact is more suitable, or even a plug-in location into which a connector engages as part of the engagement mechanism. Basically, all known technologies which are also known in the prior art can be employed for a connection between movable components.

In an alternative embodiment, provision can also be made that in the case of an adjustment capability permitting arbitrary pivot angles a fixing mechanism for the magnet unit is provided in one pivot position. It is therefore also possible to permit an arbitrary pivot position in the angular interval, in which case, however, a facility should then be provided for fixing the magnet unit relative to the housing. Compression or wraparound fixing mechanisms are particularly suitable in this situation.

In this context, at least one sliding contact and/or at least one wireless connection facility in particular are suitable as connection facilities for signal lines and power supply lines to the magnet unit, in which case what has been stated with regard to the alternative embodiment defining engagement points also applies in respect of an arbitrary pivoting capability.

It is expedient if the pivotable magnet unit has a cold head associated with the superconducting main magnet. The cold head of the main magnet can be mounted fixedly on the magnet unit and thus pivot. This is also possible without any difficulty from the weight point of view.

In order to achieve a concrete implementation of the pivoting capability, provision can be made for the magnet unit having a circular outer contour perpendicular to the pivot axis at least in the contact region with the housing to be mounted in a recess in the housing having a slide surface adapted to the outer contour which permits pivoting in the angular range. Such a type of mounting, in which case the recess can also simultaneously be used for guidance purposes, is particularly suitable in terms of a compact embodiment attaching to the housing. In this situation an alignment of outer contour and slide surface can be implemented both in the supporting region of the housing, on which the magnet unit then bears, and also on a part of the housing encompassing the magnet unit at the top.

In some embodiments, a center of gravity of the magnet unit lies on the transverse axis, in particular through the use of at least one counterweight. In this manner, a state of equilibrium exists which enables particularly easy pivoting without any imbalance. It can however be advantageous to rather facilitate slight height differences of the extremity radiography unit in different pivot positions, with for example in the above embodiment having the guide on a slide surface the pivot axis being arranged at a distance, in particular above the magnetic resonance device.

In an exemplary embodiment, provision can be made for a rotation device for rotating the housing about a vertical axis of rotation to be provided. The entire magnetic resonance device may be mounted in a rotatable fashion, which means that in particular a rotation of the housing with the magnet unit through 180° is also possible. This results in a significant space saving because it is then no longer necessary to approach the magnetic resonance device from the rear or to have space at the rear side for positioning a patient, but the rear side of the magnetic resonance device can be rotated to the front in a simple manner. This makes it possible that a patient chair associated with the magnetic resonance device can always remain on the same side of the magnetic resonance device because the magnetic resonance device can be rotated relative to the patient. Such a type of embodiment can be implemented far more simply with magnetic resonance devices for scanning an extremity (extremity scanner) than with “large” systems, since they have only a small stray field and are therefore relatively insensitive to magnetic materials in the surrounding area.

Two engagement positions of the rotation device spaced 180° apart may be provided, or a locking device for fixing the housing in a rotation position may be provided. In an exemplary embodiment, two different engagement positions may be used here that correspond to the two principally required placements of the magnetic resonance device (access to the front side or rear side). As far as connection facilities for power supply and signal lines are concerned, the statements regarding the pivot mechanism can be applied by analogy to the rotation device and the mounting of the housing, for example, in an appropriate base.

If a center of gravity of the housing lies on the axis of rotation, counterweights can be employed. It is then possible to implement rotation free from imbalance without the risk of tipping or the like.

The pivot mechanism and/or the rotation device can be driven electrically and/or be capable of manual actuation. In this situation, the implementation of a manual actuation is relatively expedient since this saves time and effort and shifts the adjustment of the magnetic resonance device into the sphere of responsibility of the operating personnel, which means that negative effects on the patient can be largely avoided. In principle, however, electrically driven adjustments are also possible. In the case of a manual actuation capability spring balancers can expediently be employed in order to permit a further simplification of the actuation. The aforementioned engagement points or engagement positions also constitute an expedient simplification in the case of the manual actuation capability.

Further embodiments may include at least one sensor for ascertaining a position of the magnet unit and a control device evaluating positions measured by the sensor are provided. If a pivot mechanism and a rotation device are present, both can suitably be provided with a sensor system so that the current position can be constantly reproduced. Such a type of sensor system can naturally also be integrated directly into electrical and/or mechanical drive means. Measured positions can be employed during operation of the magnetic resonance device for imaging purposes.

It is thus possible in a first further embodiment that the control device for ascertaining at least one imaging parameter for the magnetic resonance device is designed to be dependent on a position measured by the sensor. For example, the pivot position of the magnet unit from the horizontal gives a clear indication as to the side from which the extremity is introduced into the extremity radiography unit, which means that conclusions can be drawn therefrom regarding the orientation of the extremity to be scanned. In particular, an imaging parameter set in the form of a scan program can be assigned in each case to the different positions, which means for example that scan programs for scanning a right knee and a left knee can be correctly assigned on the basis of the detected position. Time and effort are thereby saved when adjusting the magnetic resonance device and sources of error are avoided.

Another exemplary embodiment, which can naturally also be employed additionally for ascertaining imaging parameters, is given if the magnetic resonance device has a controllable shim device for improving the homogeneity in the imaging region, in which case the control device for adjusting the shim device is designed to be dependent on a position of the magnet unit adjusted by means of the pivot mechanism and/or the rotation device. Because the homogeneity in the homogeneity region, in other words the imaging region, is also heavily dependent on materials in the surrounding area, the adjustment by means of the pivot mechanism and where applicable the rotation device can advantageously be taken into consideration according to the invention during shimming of the magnetic field. Appropriate controllable (active) shim devices are already known, for example under the name E-Shim. Shim coils are mostly employed in this situation. In this embodiment, it is therefore possible to control the shim device such that optimal shimming takes place in the magnet unit in each case during data recording. In this case, the control device for adjusting the shim device may be designed to take into consideration a characteristic curve and/or a characteristic diagram and/or a stored control parameter set for at least one position of the magnet unit. Such types of data and relationships can be obtained for example in calibration measurements which are produced during installation of the magnetic resonance device. With regard to engagement points and where applicable engagement positions, it is appropriate to the carry out the measurements in each case for these clearly defined positions and then to use the corresponding adjustments for homogenization purposes in the imaging region. With regard to an arbitrary adjustment capability, it can be expedient to interpolate between measuring points.

### BRIEF DESCRIPTION

Further advantages and details of the present invention will emerge from the exemplary embodiments described in the following and with reference to the drawings, wherein:

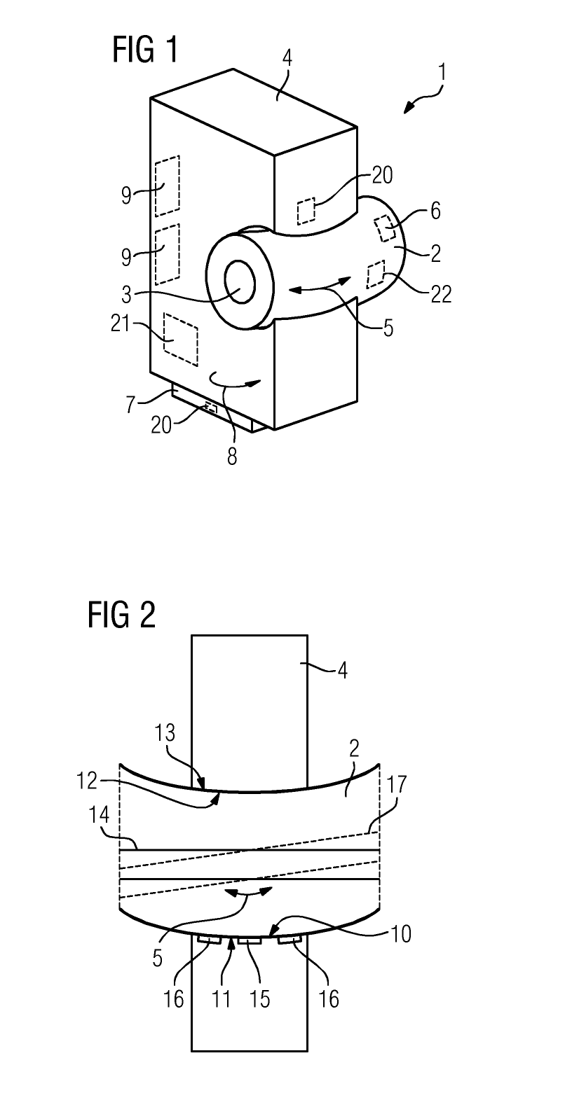

FIG. 1 shows a schematic diagram of an embodiment of a magnetic resonance device;

FIG. 2 schematically illustrates the pivoting capability of the magnet unit; and

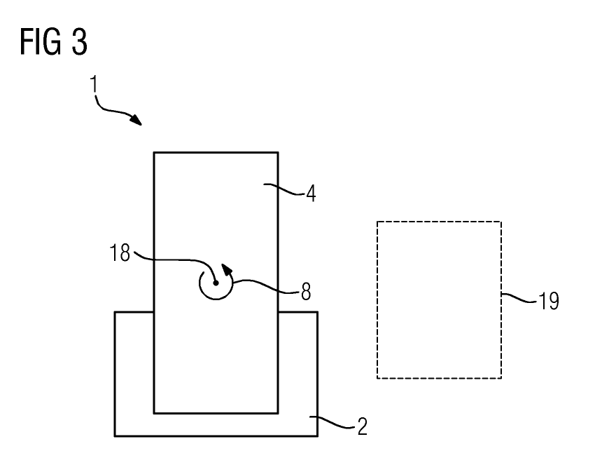

FIG. 3 schematically illustrates the rotation capability of the housing.

### DETAILED DESCRIPTION

FIG. 1 shows an exemplary embodiment of a magnetic resonance device 1. This is designed for scanning an extremity of a patient, for which purpose it has a magnet unit 2 having an extremity radiography unit 3 into which an extremity, for example an arm or a leg, of a patient can be introduced while the remainder of the patient remains outside the extremity radiography unit 3. The magnet unit 2, which observes the conventional design specifications for magnetic resonance devices, in particular therefore comprises a here superconducting main magnet having a field strength >1 Tesla, a gradient coil arrangement and a high-frequency coil arrangement, is mounted to be capable of pivoting about a horizontal pivot axis in a housing 4 which contains further components of the magnetic resonance device, cf. arrow 5. A cold head 6 only suggested here is also part of the magnet unit in this situation.

The housing 4 itself is mounted on a base 7 such that it is capable of rotation as a whole about a vertical axis, cf. arrow 8. In order to achieve simple, manual operation the center of gravity of the housing 4 with the magnet unit 2 lies on the vertical axis of rotation; counterweights 9 are provided in order to achieve this.

By way of the pivoting capability of the magnet unit 2 and the rotation capability of the housing 4 the magnetic resonance device can be brought into different positions which are conducive to comfort during an examination, although as can be seen the magnet unit 2 is not arranged centrally in the housing 4 but toward the edge thereof, in FIG. 1 toward the right-hand edge.

FIG. 2 shows a more detailed view of said pivoting capability of the magnet unit 2 in a schematic diagram. The magnet unit 2 is, as already mentioned, mounted in the housing 4. There the magnet unit 2 bears with a contact surface on a slide surface 11 on the housing 4, in which case the magnet unit 2 also fits on its upper side into the housing 4 by way of corresponding surfaces 12, 13. FIG. 2 shows a section through a vertical plane containing the longitudinal axis of the extremity radiography unit 3, in which plane the outer contour of the magnet unit 2 as can be seen, at least in the possible contact region, is designed to be round in the form of a circle in order to permit a corresponding sliding pivoting about a pivot axis lying outside the region illustrated by FIG. 2, which stands perpendicular on the image plane of FIG. 2 and consequently runs horizontally and perpendicular to the longitudinal axis of the extremity radiography unit 3. This makes it possible to tilt the extremity radiography unit 3 from the horizontal, as is likewise indicated in FIG. 2. A horizontal position 14 of the extremity radiography unit 3 is shown drawn in solid lines.

Within the slide surface 11 which acts as a guide, three engagement points are provided in the present instance, namely a middle engagement point 15 assigned to the horizontal position 14 and also two outer engagement points 16 which simultaneously mark an end stop. Because the engagement points 16 correspond to pivoting movements through +10° and −10° respectively, the magnet unit can be pivoted in an angular range of 20° in total, with three fixedly provided positions being present. In addition to the horizontal position 14, the position 17 tilted through 10° associated with the right-hand engagement point 16 is also illustrated by dashed lines in FIG. 2.

A horizontal position 14 of the extremity radiography unit 3 can for example be utilized in order to scan an arm with the magnetic resonance device. For legs, it is more favorable if they are supported sloping slightly downward in the extremity radiography unit 3. One of the positions given by the engagement points 16 can then be used, for example the position 17. The decision on where the extremity is introduced depends on which side of the extremity radiography unit 3 is situated higher; this means that the pivoting capability of the magnet unit 2 makes it possible to use the magnetic resonance device 1 from both sides. Thus, when one leg is being scanned as an extremity a patient can always position the other leg in each case comfortably beside the housing 4 or the magnet unit 2 even though the housing 4 is more extended to the one side.

The engagement points 15, 16 are used not only for the definition of fixed positions 14, 17 of the magnet unit 2 but also fulfill the function of plug-in locations; this means that whenever the magnet unit 2, for example by way of an appropriate projection, engages in an indentation corresponding to an engagement point, appropriate connection facilities for signal lines and power supply lines not illustrated here in detail provide for a corresponding connection of the magnet unit 2 with the housing 4. In an exemplary embodiment in which no engagement points are provided but an arbitrary pivoting capability having a suitable fixing device is provided, sliding contacts are provided as connection facilities; in the example illustrated here the engagement points finally form plug-in locations by way of which the connections are correctly established. The use of wireless connection facilities is also conceivable, in particular for signal transmission.

If the pivoting capability of the magnet unit 2, as illustrated here, is implemented jointly with the rotation capability, cf. arrow 8 in FIG. 1, it is actually sufficient to provide the space for a patient chair or the like, or the patient chair or some other patient support device itself only on one side of the magnetic resonance device 1 because the latter can be turned through 180°, for which purpose fixed engagement positions spaced at 180° are preferably provided, similar to what is shown in FIG. 2. FIG. 3 shows the rotation capability of the magnetic resonance device 1 in more detail in a further schematic drawing. It can be seen that the rotation capability of the housing 4 with the magnet unit 2 is implemented about a central, vertical axis of rotation 18 such that a patient positioning device 19, for example a patient chair, which is only suggested here, can remain on one side of the magnetic resonance device 1 without any problems as can be seen.

Appropriate connection facilities, for example slip couplings, can also be employed with regard to the rotation capability of the housing 4 on the base 7.

As FIG. 1 additionally shows, the magnetic resonance device also has sensors 20 by way of which the position of the magnet unit 2 or of the housing 4 in respect of the pivoting capability and the rotation capability can be acquired and forwarded to a control device 21 of the magnetic resonance device 1. This data can be evaluated in different ways in the control device 21.

On the one hand the control device 21 is designed in order to ascertain at least one imaging parameter of the magnetic resonance device 1 depending on a position measured by the sensors 20, in which case for example it is possible to conclude whether a right or a left knee of the patient is currently to be examined. An appropriate measurement program can then be selected or offered to a user for selection.

In addition however the magnetic resonance device 1 in the magnet unit 2 also has a controllable shim device 22, for example comprising shim coils, by way of which the homogeneity in the imaging region situated within the extremity radiography unit 3 can be improved. If the magnetic resonance device 1 is being set up for the first time, the effects of the pivoting and rotation operations on the homogeneity in the imaging region are measured, for example within the scope of a calibration process, so that suitable control parameter sets for achieving a maximum degree of homogeneity in each of these positions can be ascertained and stored. During operation of the magnetic resonance device 1 it is then possible to select a suitable control parameter set, depending on the data from the sensors 20. If a continuous and/or an arbitrary rotation capability and/or pivoting capability is implemented in another exemplary embodiment, characteristic curves and/or characteristic diagrams are also conceivable in order to be able to locate suitable control parameters for the shim device 22.

It should finally also be noted that with regard to the exemplary embodiment described here a manual pivoting capability/rotation capability of the magnetic resonance device 1 is provided, which can be made possible by means of suitable spring balancers and the like in addition to the aforementioned counterweights. It should however be noted in this situation that exemplary embodiments having electrically driven pivoting and/or rotation capabilities are naturally also conceivable, for which purpose suitable drive means are to be provided.

Although the invention has been illustrated and described in detail by means of the preferred exemplary embodiment, the invention is not restricted by the disclosed examples and other variations can be derived therefrom by the person skilled in the art without departing from the scope of protection of the invention.

1. A magnetic resonance device for scanning an extremity of a patient comprising:
a housing and an extremity radiography unit containing an imaging region, into which the extremity is introduced when the patient is situated outside the extremity radiography unit, in which case the extremity radiography unit is arranged along a horizontal transverse axis perpendicular to a longitudinal axis of the extremity radiography unit toward a side of the housing;
wherein a magnet unit forming the extremity radiography unit is configured to be capable of pivoting around a horizontal pivot axis perpendicular to the longitudinal axis by means of a pivot mechanism at a certain angular interval located about a horizontal situation of the longitudinal axis.

2. The magnetic resonance device as claimed in claim 1, wherein the pivot mechanism comprises a guiding assembly having a plurality of engagement points for a plurality of different pivot positions.

3. The magnetic resonance device as claimed in claim 2, wherein the plurality of engagement points are present for a central position in which the longitudinal axis lies in the horizontal plane and for at least two inclined positions lying in particular at an edge of the angular interval.

4. The magnetic resonance device as claimed in claim 1, wherein in the case of an adjustment capability permitting arbitrary pivot angles, a fixing mechanism for the magnet unit is provided in one pivot position.

5. The magnetic resonance device as claimed in claim 1, wherein the angular interval is 20°.

6. The magnetic resonance device as claimed in claim 1 wherein the magnet unit having a circular outer contour perpendicular to the pivot axis at least in a contact region with the housing is mounted in a recess in the housing having a slide surface adapted to the circular outer contour which permits pivoting in the angular range.

7. The magnetic resonance device as claimed in claim 1, further comprising a rotation device for rotating the housing about a vertical axis of rotation.

8. The magnetic resonance device as claimed in claim 7, further comprising two engagement positions of the rotation device spaced 180° apart are provided or a locking device for fixing the housing in a rotation position.

9. The magnetic resonance device as claimed in claim 7, wherein a center of gravity of the housing lies on the axis of rotation through a use of at least one counterweight.

10. The magnetic resonance device as claimed in claim 1, wherein the pivot mechanism and/or the rotation device is driven electrically and/or be capable of manual actuation.

11. The magnetic resonance device as claimed in claim 1, further comprising at least one sensor for ascertaining a position of the magnet unit and a control device evaluating positions measured by the sensor.

12. The magnetic resonance device as claimed in claim 11, wherein the control device for ascertaining at least one imaging parameter for the magnetic resonance device is configured to be dependent on a position measured by the sensor.

13. The magnetic resonance device as claimed in claim 11, wherein the magnetic resonance device has a controllable shim device for improving the homogeneity in the imaging region, in which case the control device for adjusting the shim device is configured to be dependent on a position of the magnet unit adjusted by means of the pivot mechanism and/or the rotation device.

14. The magnetic resonance device as claimed in claim 13, wherein the control device for adjusting the shim device is configured to take into consideration a characteristic curve and/or a characteristic diagram and/or a stored control parameter set for at least one position of the magnet unit.

15. The magnetic resonance device of claim 1, wherein the extremity of the patient is a leg.
